(19) United States
(12) Plant Patent Application Publication
(10) Pub. No.: US 2006/0085878 P1
(43) Pub. Date: Apr. 20, 2006

(54) Eastern redbud plant named 'Hearts of Gold'

(50) CERCIS CANADENSIS; EASTERN REDBUD

(76) Inventor: Jon L. Roethling, Raleigh, NC (US)

Correspondence Address: MYERS BIGEL SIBLEY & SAJOVEC, PO BOX 37428, RALEIGH, NC, 27627, US

(21) Appl. No.: 10/967,697

(22) Filed: Oct. 15, 2004

(51) Int. Cl. A01H 5/00

(52) U.S. Cl. PLT/216

(57) ABSTRACT

‘Hearts of Gold’ is a distinctive variety of Sercis canadensis, which is characterized by its distinct bright golden foliage compared to other cultivars of Sercis canadensis. In addition, one-year-old whips of ‘Hearts of Gold’ produce flower buds, whereas other cultivars of Sercis canadensis generally only produce flowers on older plants. Further, first-year budded ‘Hearts of Gold’ trees appear to be more vigorous and uniform in growth than similar-aged trees of other well-known cultivars.

### LATIN NAME OF THE GENUS AND SPECIES

The Latin name of the novel variety disclosed herein is Cercis canadensis var. canadensis ‘Hearts of Gold’.

### VARIETY DENOMINATION

The inventive variety of Sercis canadensis disclosed herein has been given the variety denomination ‘Hearts of Gold’.

### BACKGROUND OF THE INVENTION

The genus Cercis encompasses approximately eight species of deciduous, woody trees native to temperate portions of the eastern and western United States, eastern and central Asia, and the Mediterranean basin. Cercis is a member of the legume family (Fabaceae, or Leguminosae), within which it belongs to subfamily Caesalpinioideae, section Cercideae. Within this family, there are no other genera that are closely related to Cercis, and Cercis is believed to represent a Tertiary relict derived from a now-extinct ancestral genus.

Sercis canadensis can be found from Maine and southern Canada, west to Michigan, Illinois, Iowa, and Nebraska, where it is referred to as Sercis canadensis var. Canadensis and has the common name eastern redbud. On the southern end of its range, Sercis canadensis is native from Florida to Texas and south into northeastern Mexico. Plants found in north-central Texas and Oklahoma are referred to as Sercis canadensis var. texensis (formerly known as Cercis reniformis), commonly called Texas redbud. Plants occurring in southern Texas, northeastern Mexico, and New Mexico are known as Sercis canadensis var. mexicana, commonly called Mexican redbud. In the western range of Arizona, Utah, Nevada, and California, these plants are called Sercis canadensis var. orbiculata (recognized at the specific level as Cercis orbiculata) or commonly referred to as Arizona redbud. Until recently, plants growing in California have been recognized as Cercis occidentalis (western redbud), but this view is now being abandoned among legume taxonomists.
Parentage: ‘Hearts of Gold’ was discovered in the spring of 2002 as a seedling growing under a cultivated (garden) setting at a private residence in Greensboro, N.C. and the parentage is unknown.
Asexual reproduction: The new variety ‘Hearts of Gold’ was first asexually propagated by grafting budwood at Hidden Hollow Nursery, Belvidere, Tenn. in August 2002. The characteristics of the new cultivar have remained stable and true to type through successive asexual propagations by grafting.

### SUMMARY OF THE INVENTION

‘Hearts of Gold’ is a distinctive variety of Sercis canadensis, which is characterized by its unique bright golden foliage color as compared with other Sercis canadensis cultivars. In addition, one-year-old whips of ‘Hearts of Gold’ produce flower buds, whereas other cultivars of Sercis canadensis generally only produce flowers on older plants. Further, first-year budded ‘Hearts of Gold’ trees appear to be more vigorous and uniform in growth than similar-aged trees of other well-known cultivars.

### BRIEF DESCRIPTION OF THE DRAWINGS

The following figures are photographs of 1-year-old whips of ‘Hearts of Gold’ budded onto 2-year-old understock. The trees are field-grown with irrigation.

FIG. 1 illustrates the stem and foliage color of ‘Hearts of Gold’.

FIG. 2 illustrates the stem and foliage color of ‘Hearts of Gold’ and, in particular, shows the red tinge present on the new flush of growth.

FIG. 3 illustrates the foliage color of ‘Hearts of Gold’ (right) compared to a Sercis canadensis ‘Forest Pansy’ (left; unpatented).

FIG. 4 illustrates the foliage color and form of ‘Hearts of Gold’ trees (center) as compared with Sercis canadensis ‘Forest Pansy’ (purple foliaged trees, left) and Sercis canadensis ‘Appalachia Red’ (green foliaged trees, right; unpatented).

FIG. 5 shows an aerial view of the foliage color and form of ‘Hearts of Gold’ plants (center) compared to Sercis canadensis ‘Forest Pansy’ (purple foliage, right) and Sercis canadensis ‘Appalachia Red’ (green foliage, left).

The photographs in the figures show the colors as true as reasonably possible by digital photography. Colors in the photographs may differ slightly from the color values cited in the detailed botanical description, which accurately describe the colors of the new eastern redbud ‘Hearts of Gold’.

### DETAILED BOTANICAL DESCRIPTION OF THE VARIETY

The following is a detailed botanical description of a new and distinct variety of Sercis canadensis known as ‘Hearts of Gold’ based upon observations of one-year-old whips grown in Belvidere, Tennessee and the original six- to seven-year-old founder plant grown in Greensboro, N.C.

Those skilled in the art will appreciate that certain characteristics will vary with older or, conversely, with younger plants. ‘Hearts of Gold’ has not been observed under all possible environmental conditions. Where dimensions, sizes, colors and other characteristics are given, it is to be understood that such characteristics are approximations or averages set forth as accurately as practicable. The phenotype of the variety may differ from the descriptions set forth herein with variations in environmental, climactic and cultural conditions. Color notations are based on The Royal Horticultural Society Colour Chart, The Royal Horticultural Society, London, 2001 except where general color terms are used.

‘Hearts of Gold’ is a small deciduous tree of Sercis canadensis useful for landscaping. ‘Hearts of Gold’ has distinct, bright golden foliage which is unusual for Sercis canadensis as these plants generally have medium-to-dark green leaves unless there is a nutrient deficiency. A botanical description of ‘Hearts of Gold’ and comparisons with other varieties of Sercis canadensis are provided below. Unless otherwise indicated, ‘Hearts of Gold’ exhibits traits which are typical of other cultivars of Sercis canadensis var. Canadensis.

### Technical Description of the Variety

Plant form/size: When left untrained, ‘Hearts of Gold’ will form a small tree (approximately 20-feet tall) at maturity with upright stems and primary branches, wherein the smaller branches arch to produce a vase- to rounded-shaped canopy.
Stem: Dark brown to dark, gray-brown, smooth, and prominently covered with tiny, gray lenticels. With age, stems will develop a scaly, gray bark, reddish-brown in color underneath.
Branches: Two-year-old branches are slender, dark brown to dark, gray-brown with gray lenticels. Current season growth bears internodes ranging in length from ˜1 to 2.5 inches (˜3 to 6 cm).
Leaves: Deciduous, falling in the autumn of the year. Leaves are broadly ovate in shape, with pronounced cordate leaf bases and acute leaf apices. Margins on all leaves are entire. Petioles range from ˜1 to 2.5 inches (˜3 to 6 cm) long. Mature leaf blades measure ˜3 to 5 inches (˜8 to 12 cm) long by ˜3 to 5 inches (˜8 to 12 cm) wide. Young, vigorous plants of ‘Hearts of Gold’ produce leaves with dimensions measuring ˜6 inches (˜15 cm) long by ˜7 inches (˜18 cm) wide. Leaf color is vivid, bright, yellow-green (Yellow-Green Group 151). Leaves on an individual branch vary in color only slightly with age. From the newest (subapical) leaf to those occurring distally along the branch, leaf color can range through all subgroups of the Yellow-Green Group 151 (151A through 151D). Under conditions of bright sunlight, foliage in nonwater-stressed plants retains bright golden color that grades to chartreuse-yellow over the course of the growing season. Under shaded growing conditions, including those imparted by the tree canopy, leaves will grade from bright golden to chartreuse-yellow to green. However, even under these shaded conditions, plants retain the golden leaf color on the outermost leaves of the tree canopy. Golden foliage color is not affected by soil fertility or plant nutrient status. The unique color is not caused by lack of any known essential plant element, including iron, magnesium, or nitrogen.
Flowers: Pea-like in configuration, ˜0.43 inch (˜11 mm) long (measured from keel to standard), borne in clusters of ˜6 to 8 flowers on older branches. Measured from the apical end of the peduncle to the apex of the keel, flower length averages ˜0.44 inch (˜11.3 mm) long (N=10). Peduncle length averages ˜0.42 inch (˜10.7 mm) (N=10). In Guilford County, N.C., flowers appear in late March to early April and last for approximately 2 to 3 weeks. Flowers are produced in great abundance, both at the nodes of previous years' wood, as well as along the older trunks (cauliflory). Flower clusters contain an average of 6.6 flowers (N=15) per axillary cluster. These flowers are produced even on young plants, as observed from field-grown, vegetatively propagated (grafted) plants. Standard or banner petal color and keel petal color are Purple Group 78B. Wing petals are Purple Group 77B. Calyx color also varies. Calyx lobes are Red-Purple Group 74C. The base of the fused portion of the calyx is Red-Purple Group 71A. No flower fragrance of any significance is noted for ‘Hearts of Gold’.
Fruits: Thin, flattened, elongated legumes, measuring ˜2 to 4 inches (˜5 to 10 cm) long by ˜0.4 to 0.5 inches (˜10 to 13 mm) wide. Legumes are borne along the branches in a pendent manner, are reddish-brown in color, and become near black at maturity. Legumes can persist on the tree through the winter months.
Growth rate: Moderate to rapid when young. One-year-old budded plants produce approximately 6- to 7-foot tall plants within one growing season. Older plants will grow at the rate of ˜1 to 2 feet per year, and established specimens will grow at a further reduced rate.
Hardiness: ‘Hearts of Gold’ is hardy to USDA Cold Hardiness Zone 5.
Disease and pest resistance/susceptibility: Disease and pest resistance/susceptibility for ‘Hearts of Gold’ is the same as for the species.

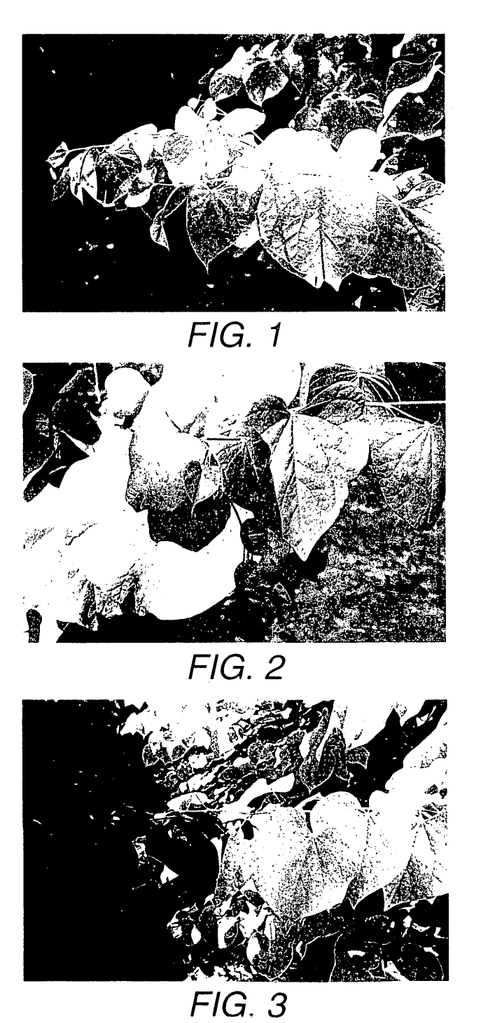

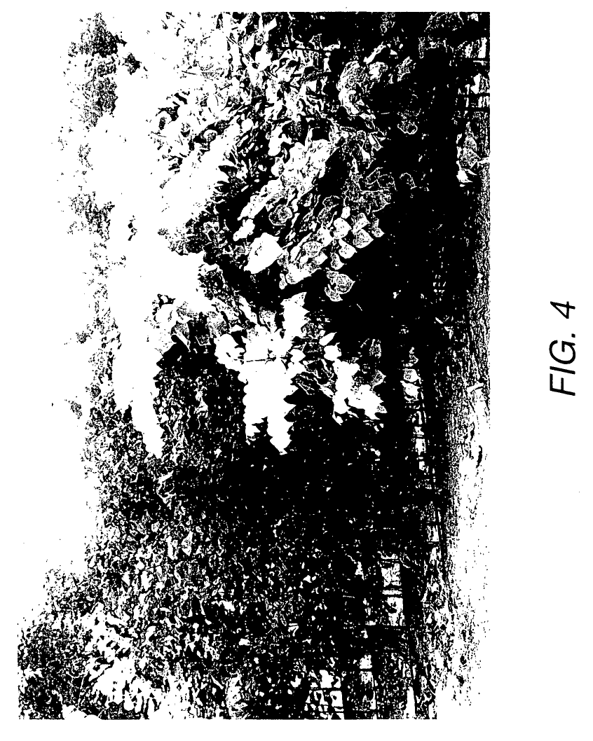

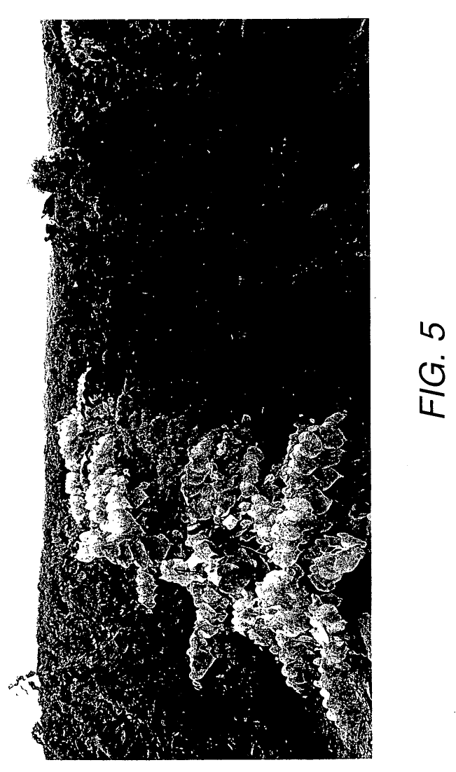

These and other features and characteristics of ‘Hearts of Gold’ are apparent from FIGS. 1-5.

### Comparisons with Other Sercis canadensis

‘Hearts of Gold’ is distinguishable from other known cultivars of Cercis canadensis and all other Cercis taxa by the foliage color. Mature leaves of typical specimens of Sercis canadensis are dark blue-green and dull on the upper surface, lighter beneath. On new growth, most plants of Cercis canadensis bear light green, red-tinted leaves, whereas the new growth on ‘Hearts of Gold’is more golden in color with a red tint. On both the upper and lower leaf surfaces, new foliage color in a typical Sercis canadensis specimen will be light green (Yellow-Green Group 144A). In contrast, Cercis canadensis ‘Hearts of Gold’ bears vivid bright, yellow-green leaves (Yellow-Green Group 151) from the newest leaf to those occurring distally along the branch (see, e.g., FIG. 3). Further, ‘Hearts of Gold’ flower buds, as observed in Belvidere, Tenn., are produced on one-year-old whips, whereas other cultivars of Sercis canadensis do not exhibit this trait and typically produce buds only after two to three years. Moreover, first-year budded ‘Hearts of Gold’ trees appear to be more vigorous and uniform in growth than similar-aged trees of other well-known cultivars of Cercis canadensis, such as ‘Forest Pansy’, ‘Appalachia Red’, ‘Covey’ (U.S. Plant Pat. No. 10,328) and ‘Tennessee Pink’ (unpatented).

That which is claimed is:

1. A new and distinct variety of Sercis canadensis plant named ‘Hearts of Gold’, substantially as described and illustrated herein.
